(19) United States
(12) Patent Application Publication
(10) Pub. No.: US 2019/0142029 A1
(43) Pub. Date: May 16, 2019

(54) CLEAN LABEL WHEAT PROTEIN ISOLATE

(71) Applicant: Manildra Milling Corporation, Leawood, KS (US)

(72) Inventors: Brook Carson, Prairie Village, KS (US); Neal Bassi, Overland Park, KS (US); Luke Stockstell, Olathe, KS (US)

(21) Appl. No.: 16/188,755

(22) Filed: Nov. 13, 2018

Related U.S. Application Data
(60) Provisional application No. 62/585,345, filed on Nov. 13, 2017.

(51) Int. Cl. A23J 3/34; A23J 1/12; A23J 3/18; A23L 33/185; C12P 21/06

(52) U.S. Cl. CPC A23J 3/346; A23J 1/125; A23J 3/18; A23V 2300/28; C12P 21/06; A23V 2002/00; A23V 2250/5486; A23L 33/185

(57) ABSTRACT

A method of preparing a functionalized wheat protein product is provided. The method comprises an enzyme treatment step wherein a wheat protein composition is contacted with a primary enzyme, and optionally with a secondary enzyme. The primary enzyme comprises a protease that is naturally occurring in a fruit.

### CROSS-REFERENCE TO RELATED APPLICATIONS

This application claims priority to U.S. Provisional Patent Application Ser. No. 62/585,345, filed on Nov. 13, 2017, to Brook Carson, Neal Bassi, and Luke Stockstell, entitled “Clean Label Wheat Protein Isolate,” currently pending, the entire disclosure of which is incorporated herein by reference.

### BACKGROUND

Consumer interest in “clean label” food products has been rapidly rising in recent years. While precise definitions can vary, the term “clean label” generally encompasses food products made exclusively from natural ingredients that are familiar to most consumers. Stated another way, “clean label” food products are generally free of artificial ingredients and synthetic chemicals. Products meeting these criteria are viewed favorably by consumers and are often able to command a premium price in the marketplace.

Simultaneously, there is also increasing consumer interest in foods that are high in protein. Commonly used examples of processed proteins include pea, rice, whey, milk, soy, and wheat. Other commonly used whole ingredients that provide protein include beans, nuts, and grains. Unfortunately, when proteins are added to food products, and especially to bakery products, the integrity of the finished product—such as its flavor, structure, and texture—can be diminished.

With an ever-growing demand for protein sources, the market often struggles with supply, in terms of both quantity and availability. Furthermore, although many sources of protein are available in the market, there is an increasing demand for sources that meet clean label criteria.

Wheat protein is more readily available than many other sources, and can be processed to provide unique functional advantages, particularly for use in baked goods. Unfortunately, it remains challenging to consistently provide wheat protein sources that exhibit the required functional characteristics, are free of artificial ingredients, and are suitable for use in a clean label food product.

It is therefore desirable to provide a wheat protein source that is suitable for use in clean label food products, and in particular for use in clean label baked goods.

### SUMMARY OF THE INVENTION

Provided herein is a method of preparing a functionalized wheat protein product. The method comprises an enzyme treatment step wherein a wheat protein composition is contacted with a primary enzyme, and optionally with a secondary enzyme. The primary enzyme comprises a protease that is naturally occurring in a fruit.

Also provided is a functionalized wheat protein product prepared according to a method as described herein.

Other objects and features will be in part apparent and in part pointed out hereinafter.

### BRIEF DESCRIPTION OF THE DRAWINGS

For a better understanding of the various embodiments, reference may be made to the accompanying figures in which:

FIG. 1 is a mixograph for dough made with a commercially available wheat flour.

FIG. 2 is a mixograph for dough made with a commercially available wheat flour and 3% by weight of a functionalized wheat protein product as described herein.

FIGS. 3A, 3B, and 3C are mixographs for functionalized wheat protein products as described herein.

FIG. 4 is a photograph of sliced bread prepared as described in Example 17. The bread labeled “4” was prepared using functionalized wheat protein product as described herein, while the bread labeled “1” is a control prepared without added protein.

FIG. 5 is a photograph of a functionalized wheat protein product as described herein.

### DETAILED DESCRIPTION OF THE INVENTION

The invention will now be described with reference to the figures. The following detailed description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The present invention is defined by the appended claims and the description is, therefore, not to be taken in a limiting sense and shall not limit the scope of equivalents to which such claims are entitled.

Provided herein are methods of preparing a functionalized wheat protein product. In the methods provided herein, a wheat protein composition is treated with a primary enzyme comprising a protease that is naturally occurring in a fruit, thereby forming a functionalized wheat protein composition suitable for use in preparing clean label food products. Optionally, the wheat protein composition may also be treated with a secondary enzyme that further processes the wheat protein.

The wheat protein products provided herein can be used as a clean label alternative to many wheat proteins currently on the market. Among other advantages, various embodiments may be used in food applications to provide improved dough rheology, improved dough extensibility, and optimized finished product texture. The wheat proteins provided herein are particularly suitable for use in baked goods—because the proteins are wheat-based, they are able to maintain the gluten network that provides the desirable texture in many bakery products.

### Starting Material

Generally, the wheat protein composition can comprise any source of wheat protein known in the art. The wheat protein composition typically has a wheat protein content of at least about 50% by weight.

For example, in some embodiments, the wheat protein composition comprises vital wheat gluten. Typically, vital wheat gluten has a wheat protein content of at least about 60% by weight. For example, the vital wheat gluten may have a wheat protein content of at least about 65%, at least about 70%, or at least about 75% by weight. In some embodiments, the vital wheat gluten has a wheat protein content of from about 60% by weight to about 90% by weight, from about 65% by weight to about 85% by weight, or from about 70% by weight to about 80% by weight.

In other embodiments, the wheat protein composition comprises a wheat protein isolate. Typically, the wheat protein isolate has a wheat protein content of at least about 80% by weight. For example, the wheat protein isolate may have a wheat protein content of at least about 85% by weight, or at least about 90% by weight. In some embodiments, the wheat protein isolate has a wheat protein content of from about 80% by weight to about 99% by weight, from about 85% by weight to about 98% by weight, or from about 90% by weight to about 97% by weight.

### Primary Enzyme

The primary enzyme comprises a protease that is naturally occurring in a fruit. Preferably, the primary enzyme is effective to inhibit the S-S bonds that naturally form when wheat protein is hydrated.

The primary enzyme can comprise one or more components naturally occurring in pineapple. For example, the primary enzyme can comprise bromelain.

The primary enzyme can comprise one or more components naturally occurring in papaya. For example, the primary enzyme can comprise papain.

The primary enzyme can comprise one or more components naturally occurring in kiwifruit. For example, the primary enzyme can comprise actinidin.

The primary enzyme can be utilized in any available form. For example, the methods described herein may comprise using a fruit comprising the primary enzyme (e.g., blended or pureed pineapple, papaya, or kiwifruit), using a natural fruit extract comprising the primary enzyme, or using a commercially or industrially produced enzyme.

### Secondary Enzyme

The methods described herein may optionally utilize a secondary enzyme in addition to the primary enzyme. As discussed above, the primary enzyme comprises one or more proteolytic enzymes that inhibit the S-S bonds that naturally form when wheat protein is hydrated. The secondary enzyme, if present, serves to break down the wheat proteins into even smaller fragments.

In general, the primary enzyme (e.g., bromelain) can be used to produce an extensible wheat protein suitable for use in many food applications. In situations where greater solubility is required, the secondary enzyme can be utilized to further process the wheat protein and provide the desired functionality.

The secondary enzyme can comprise a protease. Non-limiting examples of suitable secondary enzymes include metallo-type and serine-type enzymes.

### Dilution Step

In some embodiments, the methods described herein comprise an initial dilution step in which the primary enzyme and optional secondary enzyme are mixed with water. Particularly where the wheat protein composition has a low moisture content, diluting the primary enzyme and optional secondary enzyme in water can enable them to more readily react with the wheat protein composition. Added water can be removed using a subsequent drying step, as explained in further detail below.

### Enzyme Treatment Step

The methods described herein comprise an enzyme treatment step in which the wheat protein composition is contacted with a primary enzyme, which may be selected as described above. In some embodiments, the enzyme treatment step further comprises contacting the wheat protein composition with a secondary enzyme, which may be selected as described above.

The wheat protein composition may be contacted with the primary and secondary enzymes simultaneously (i.e., in a single stage), or separately (i.e., in multiple stages). In embodiments where the enzyme treatment step comprises multiple stages, the wheat protein composition may be contacted with the primary enzyme followed by the secondary enzyme, or alternatively may be contacted with the secondary enzyme followed by the primary enzyme.

The enzyme treatment step can be carried out under increased temperature (i.e., a temperature in excess of room temperature). For example, the enzyme treatment step can be carried out at a temperature of at least about 40° C., at least about 50° C., or at least about 55° C. In some embodiments, the enzyme treatment step is carried out at a temperature of from about 45° C. to about 80° C., from about 55° C. to about 70° C., from about 50° C. to about 65° C., or from about 55° C. to about 60° C.

The enzyme treatment step is preferably carried out for a time sufficient for the reaction between the primary enzyme, the optional secondary enzyme, and the wheat protein to proceed substantially to completion. For example, the enzyme treatment step may be carried out for a period of at least about 15 minutes, at least about 30 minutes, at least about 40 minutes, or at least about 1 hour. In some embodiments, the enzyme treatment step is carried out for a period of from about 30 minutes to about 4 hours, from about 45 minutes to about 3 hours, or from about 1 hour to about 2 hours.

In some embodiments, the reaction mixture comprising the primary enzyme, the optional secondary enzyme, and the wheat protein is mixed during at least a portion of the enzyme treatment step. In preferred embodiments, the reaction mixture is mixed for substantially the full duration of the enzyme treatment step.

The enzyme treatment step can be carried under acidic conditions, wherein the pH of the reaction mixture is less than 7. For example, the enzyme treatment step can comprise maintaining the pH of the reaction mixture within a range of from about 3 to about 7. In preferred embodiments, the enzyme treatment step comprises maintaining the pH of the reaction mixture within a range of from about 5 to about 7, or from about 5.5 to about 6.5. In other embodiments, the enzyme treatment step comprises maintaining the pH of the reaction mixture within a range of, for example, from about 3 to about 5, or from about 3.5 to about 4.5.

### Deactivation Step

The methods described herein can further comprise an enzyme deactivation step, following completion of the enzyme treatment step, in which the primary enzyme and optional secondary enzyme are substantially deactivated.

For example, the deactivation step can comprise heating the reaction mixture comprising the primary enzyme, the optional secondary enzyme, and the wheat protein to a temperature sufficient to deactivate the primary and secondary enzymes. In some embodiments, the deactivation step comprises heating the reaction mixture to a temperature of at least about 65° C., at least about 70° C., at least about 75° C., at least about 80° C., at least about 85° C., or at least about 90° C.

The deactivation step is preferably carried out for a duration sufficient to ensure that substantially all of the primary enzyme and optional secondary enzyme is deactivated. For example, the deactivation step can be carried out for a period of at least about 2 minutes, at least about 5 minutes, or at least about 10 minutes.

### Drying Step

The methods described herein can further comprise a drying step, following completion of the enzyme treatment step, in which the reaction mixture comprising the primary enzyme, the optional secondary enzyme, and the wheat protein is dried, thereby forming a functionalized wheat protein composition suitable for use in preparing clean label food products.

In some embodiments, the method comprises an enzyme deactivation step as discussed above that is effective to dry the reaction mixture, for example by exposing the reaction mixture to an increased temperature for a sufficient duration to substantially remove the water content. Accordingly, in these embodiments, the enzyme deactivation step effectively serves as the drying step, and no further drying is required.

In other embodiments, the reaction mixture can be dried by any means known to those skilled in the art. Non-limiting examples of suitable drying methods include spray drying, freeze drying, and convective hot air drying, for example using a ring dryer, an oven dryer, or a flash dryer. In a preferred embodiment, the drying step comprises freeze drying the reaction mixture, thereby providing a functionalized wheat protein composition.

### Functionalized Wheat Protein Products

Also within the scope of the present disclosure are functionalized wheat protein products that are suitable for use in clean label food products. The functionalized wheat protein products may be prepared, for example, according to the methods described herein.

Other objects and features will be in part apparent and in part pointed out hereinafter.

### Mixing Properties

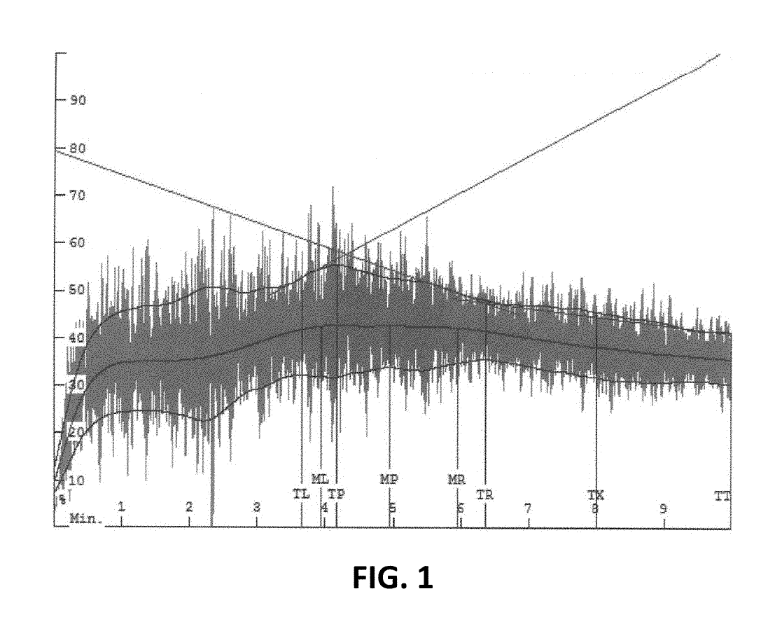

FIGS. 1, 2, 3A, 3B, and 3C are mixographs, which are used by those skilled in the art to show the mixing properties of dough compositions. The mixograph method of the American Association of Cereal Chemists (AACC) Method 54-40.02 was used and is hereby incorporated by reference. As a person of skill in the art will understand, the peaks, widths, and heights of the band or envelope indicate the time and energy required to mix the dough.

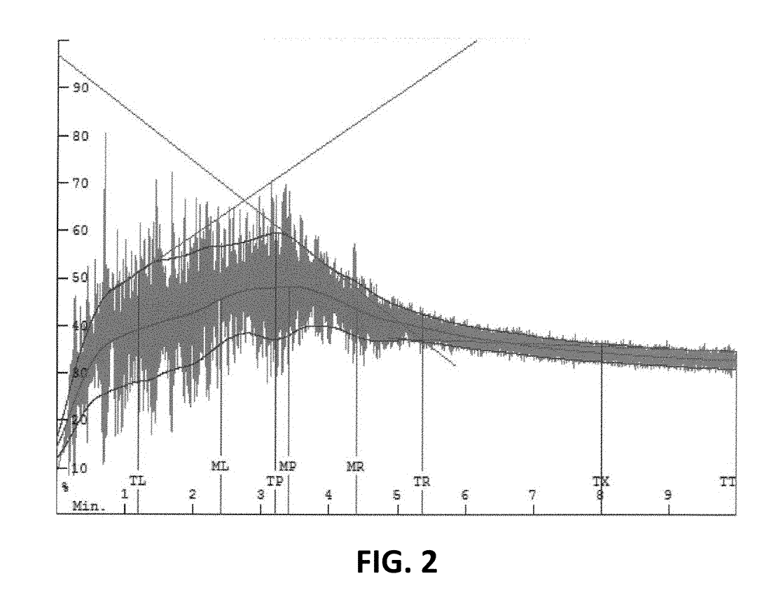

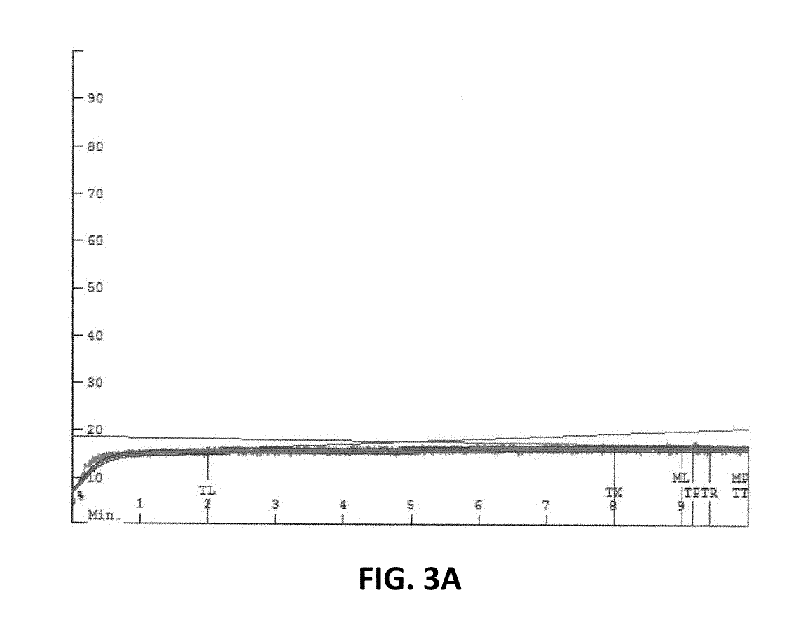

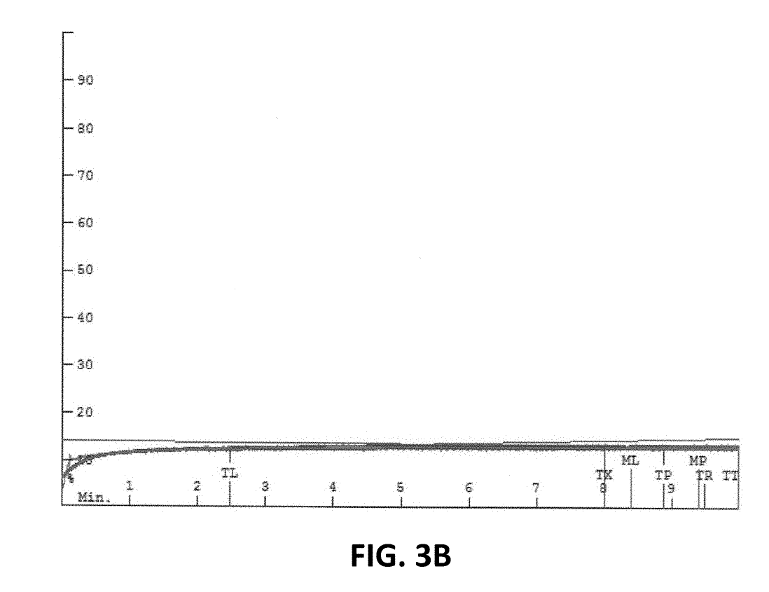

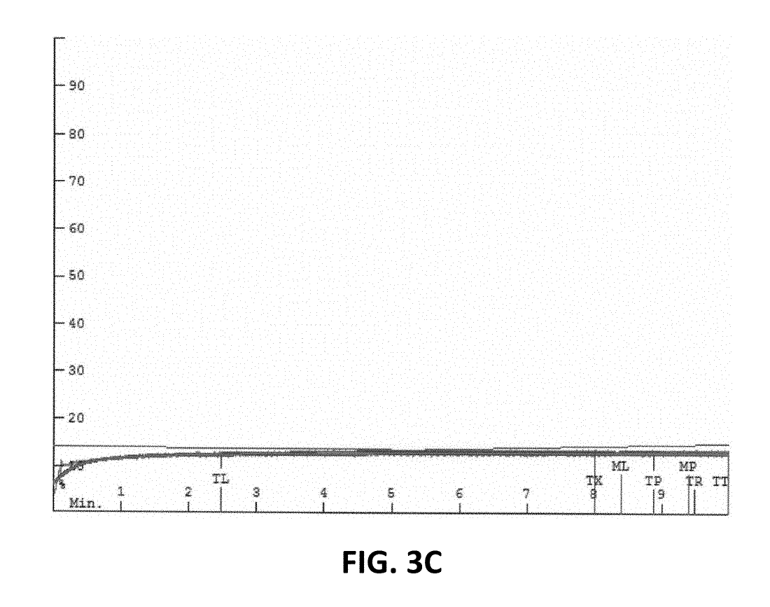

In particular, FIG. 1 is a mixograph for dough made with a commercially available wheat flour, FIG. 2 is a mixograph for dough made with a commercially available wheat flour and 3% by weight of a functionalized wheat protein product as described herein, and FIGS. 3A, 3B, and 3C are mixographs for functionalized wheat protein products as described herein.

As will be understood, relevant times in the envelopes depicted in FIGS. 1-3C are represented graphically as vertical lines which overlay the data. The line marked MP represents the midline (illustrated by the middle curve shown in green) mixing peak and the lines marked ML and MR represent points in time that are one minute before or to the left (ML) and one minute after or to the right (MR) the midline mixing peak (MP). The line marked TP represents the envelope (illustrated by the upper and lower curves shown in red) mixing peak and the lines marked TL and TR represent points in time before or to the left (TL) and after or to the right (TR) the envelope mixing peak (TP). As will further be understood, the line marked TX is an arbitrary time (in this case eight minutes) set for data collection in order to determine the width of the envelope two minutes prior to the end of the analysis (in this case 10 minutes). The line marked TT refers to curve tail and the end of the analysis.

It will be appreciated that the functionalized wheat protein products described herein may reduce the time and energy required to mix dough compositions used to prepare baked goods.

### Examples

The following non-limiting examples are provided to further illustrate the present invention.

### Example 1

A functionalized wheat protein product was prepared using the following experimental procedure. A hot plate was started, and a water bath maintained at a temperature of from 55-60° C. A portion of the 55-60° C. water (314 grams) was added to a 2000 mL beaker.

To the beaker was added 0.500 grams of a bromelain enzyme (Bromelain 240). Next, 186 grams of high protein vital wheat gluten (GemPro HPG) (6% moisture-175 grams DS) was slowly added, followed by an additional 0.375 grams of bromelain enzyme. The resulting mixture was maintained at a temperature of from 55-60° C. and a pH of from 5.5-6.5 for 1 hour under constant mixing.

The sample was then deactivated by raising its temperature to 75° C. The deactivated sample was then freeze dried to produce the final product.

### Example 2

A functionalized wheat protein product was prepared using the following experimental procedure. A hot plate was started, and a water bath maintained at a temperature of from 55-60° C. A portion of the 55-60° C. water (628 grams) was added to a 2000 mL beaker.

To the beaker was added 0.700 grams of bromelain enzyme. Next, 372 grams of high protein vital wheat gluten (6% moisture-350 grams DS) was slowly added, followed by an additional 0.350 grams of bromelain enzyme. The resulting mixture was maintained at a temperature of from 55-60° C. and a pH of from 5.5-6.5 for 1 hour under constant mixing.

The sample was then deactivated by raising its temperature to 75° C. The deactivated sample was then freeze dried to produce the final product.

### Example 3

A functionalized wheat protein product was prepared using the following experimental procedure. A hot plate was started, and a water bath maintained at a temperature of from 55-60° C. A portion of the 55-60° C. water (628 grams) was added to a 2000 mL beaker.

To the beaker was added 0.700 grams of bromelain enzyme, followed by 0.350 grams of a xylanase enzyme (Rhohalase SEP VISCO). Next, 372 grams of high protein vital wheat gluten (6% moisture-350 grams DS) was slowly added, followed by an additional 0.305 grams of bromelain enzyme. The resulting mixture was maintained at a temperature of from 55-60° C. and a pH of from 5.5-6.5 for 1 hour under constant mixing.

The sample was then deactivated by raising its temperature to 75° C. The deactivated sample was then freeze dried to produce the final product.

### Example 4

A functionalized wheat protein product was prepared using the following experimental procedure. A hot plate was started, and a water bath maintained at a temperature of from 55-60° C. A portion of the 55-60° C. water (795 grams) was added to a 2000 mL beaker.

To the beaker was added 1.00 grams of bromelain enzyme. Next, 372 grams of high protein vital wheat gluten (6% moisture-350 grams DS) was slowly added, followed by an additional 0.750 grams of bromelain enzyme. The resulting mixture was maintained at a temperature of from 55-60° C. and a pH of from 5.5-6.5 for 2 hours under constant mixing.

The mixture was then split into two portions. A first portion was mixed for an additional hour, after which the pH was lowered to 3.8 through addition of sulfuric acid. The first portion was then deactivated by raising its temperature to 75° C. The second portion was immediately lowered to a pH of 3.8 through addition of sulfuric acid, and then deactivated by raising its temperature to 75° C. for 10 minutes.

### Example 5

A functionalized wheat protein product was prepared using the following experimental procedure. A hot plate was started, and a water bath maintained at a temperature of from 55-60° C. A portion of the 55-60° C. water (795 grams) was added to a 2000 mL beaker.

To the beaker was added 1.00 grams of bromelain enzyme. Next, 372 grams of high protein vital wheat gluten (6% moisture-350 grams DS) was slowly added, followed by an additional 0.750 grams of bromelain enzyme. The resulting mixture was maintained at a temperature of from 55-60° C. and a pH of from 5.5-6.5 for 4 hours under constant mixing.

The pH of the sample was then adjusted to 4.5 by addition of sulfuric acid. The sample was then centrifuged with the intention of saving the supernatant for subsequent testing. It was noted, however, that the sample did not separate with centrifuging.

### Example 6

A functionalized wheat protein product was prepared using the following experimental procedure. A hot plate was started, and a water bath maintained at a temperature of from 60-65° C. A portion of the 60-65° C. water (795 grams) was added to a 2000 mL beaker.

To the beaker was added 2.00 grams of bromelain enzyme. Next, 372 grams of high protein vital wheat gluten (6% moisture-350 grams DS) was slowly added, followed by an additional 1.50 grams of bromelain enzyme. The resulting mixture was maintained at a temperature of from 55-60° C. and a pH of from 5.5-6.5 for 2 hours under constant mixing.

### Example 7

A functionalized wheat protein product was prepared using the following experimental procedure. A hot plate was started, and a water bath maintained at a temperature of from 55-60° C. A portion of the 55-60° C. water (226.6 grams) was added to a 2000 mL beaker.

To the beaker was added 0.300 grams of bromelain enzyme. Next, 106.4 grams of high protein vital wheat gluten (6% moisture-175 grams DS) was slowly added, followed by an additional 0.200 grams of bromelain enzyme. The resulting mixture was maintained at a temperature of from 55-60° C. and a pH of from 5.5-6.5 for 2 hours under constant mixing.

The pH of the sample was lowered to 3.8 using sulfuric acid, and the sample was then deactivated by raising its temperature to 75° C. The deactivated sample was then freeze dried to produce the final product.

### Example 8

A functionalized wheat protein product was prepared using the following experimental procedure. A hot plate was started, and a water bath maintained at a temperature of from 55-60° C. A portion of the 55-60° C. water (226.6 grams) was added to a 2000 mL beaker.

To the beaker was added 0.300 grams of bromelain enzyme. Next, 106.4 grams of high protein vital wheat gluten (6% moisture-175 grams DS) was slowly added, followed by an additional 0.200 grams of bromelain enzyme. The resulting mixture was maintained at a temperature of from 55-60° C. and a pH of from 5.5-6.5 for 2 hours under constant mixing.

The pH of the sample was lowered to 3.8 using sulfuric acid, and the sample was then deactivated by raising its temperature to 75° C. The deactivated sample was then freeze dried to produce the final product.

### Example 9

A functionalized wheat protein product was prepared using the following experimental procedure. A hot plate was started, and a water bath maintained at a temperature of from 55-60° C. A portion of the 55-60° C. water (130 grams) was added to a 2000 mL beaker.

To the beaker was added 2.00 grams of bromelain enzyme, followed by 0.100 grams of a protease. Next, 70 grams of high protein vital wheat gluten was slowly added. The resulting mixture was maintained at a temperature of from 55-60° C. and a pH of from 5.5-6.5 for 1 hour under constant mixing.

An additional 0.1 grams of a protease was then added, and the resulting mixture was maintained at a temperature of from 55-60° C. and a pH of from 5.5-6.5 for an additional 1 hour under constant mixing.

The sample was then deactivated by raising its temperature to 90° C. for 10 minutes. The deactivated sample was then tested in glaze, aeration, and dough.

### Example 10

A functionalized wheat protein product was prepared using the following experimental procedure. A hot plate was started, and a water bath maintained at a temperature of from 55-60° C. A portion of the 55-60° C. water (130 grams) was added to a 2000 mL beaker.

To the beaker was added 1 grams of concentrated bromelain enzyme, followed by 0.100 grams of a protease. Next, 70 grams of high protein vital wheat gluten was slowly added. The resulting mixture was maintained at a temperature of from 55-60° C. and a pH of from 5.5-6.5 for 1 hour under constant mixing.

An additional 0.1 grams of a protease was then added, and the resulting mixture was maintained at a temperature of from 55-60° C. and a pH of from 5.5-6.5 for an additional 1 hour under constant mixing.

The sample was then deactivated by raising its temperature to 90° C. for 10 minutes. The deactivated sample was then tested in glaze, aeration, and dough.

### Example 11

A functionalized wheat protein product was prepared using the following experimental procedure. A hot plate was started, and a water bath maintained at a temperature of from 55-60° C. A portion of the 55-60° C. water (130 grams) was added to a 2000 mL beaker.

To the beaker was added 1.00 grams of bromelain enzyme, followed by 0.0500 grams of a protease. Next, 70 grams of high protein vital wheat gluten was slowly added. The resulting mixture was maintained at a temperature of from 55-60° C. and a pH of from 5.5-6.5 for 1 hour under constant mixing.

At this time a small sample of the mixture was taken for use in a whip test. An additional 0.05 grams of a protease was then added to the remaining mixture, which then was maintained at a temperature of from 55-60° C. and a pH of from 5.5-6.5 for an additional 1 hour under constant mixing.

The sample was then deactivated by raising its temperature to 90° C. for 10 minutes. The deactivated sample was then tested in glaze, aeration, and dough.

### Example 12

A functionalized wheat protein product was prepared using the following experimental procedure. A hot plate was started, and a water bath maintained at a temperature of from 55-60° C. A portion of the 55-60° C. water (130 grams) was added to a 2000 mL beaker.

To the beaker was added 0.100 grams of a protease. Next, 70 grams of Freeze dried natural GP 3300 wheat protein isolate was slowly added. The resulting mixture was maintained at a temperature of from 55-60° C. and a pH of from 5.5-6.5 for 1 hour under constant mixing.

The sample was then deactivated by raising its temperature to 90° C. for 10 minutes. The deactivated sample was then tested in glaze, aeration, and dough.

### Example 13

A functionalized wheat protein product was prepared using the following experimental procedure. A hot plate was started, and a water bath maintained at a temperature of from 55-60° C. A portion of the 55-60° C. water (130 grams) was added to a 2000 mL beaker.

To the beaker was added 0.14 grams of bromelain enzyme, followed by 0.0300 grams of a protease. Next, 70 grams of high protein vital wheat gluten was slowly added. The resulting mixture was maintained at a temperature of from 55-60° C. and a pH of from 5.5-6.5 for 1 hour under constant mixing.

An additional 0.03 grams of a protease was then added to the remaining mixture, which then was maintained at a temperature of from 55-60° C. and a pH of from 5.5-6.5 for an additional 1 hour under constant mixing.

The sample was then deactivated by raising its temperature to 90° C. for 10 minutes. The deactivated sample was then tested in glaze, aeration, and dough.

### Example 14

A functionalized wheat protein product was prepared using the following experimental procedure. A hot plate was started, and a water bath maintained at a temperature of from 55-60° C. A portion of the 55-60° C. water (195 grams) was added to a 2000 mL beaker. A high speed, homogenizing mixer was used to mix the contents of the beaker during the following addition steps.

To the beaker was added 0.788 grams of bromelain enzyme, followed by 0.0116 grams of a protease. Next, 105 grams of high protein vital wheat gluten was slowly added. The resulting mixture was maintained at a temperature of from 55-60° C. and a pH of from 5.5-6.5 for 1 hour under constant mixing.

The sample was then deactivated by raising its temperature to 90° C. for 10 minutes. The deactivated sample was then freeze dried.

### Example 15

A bread product was prepared using a functionalized wheat protein as described herein. For comparison, a control bread product was prepared using the same recipe, except without any added protein.

Formulation #

| | 1 (Control) | 4 (Experimental) |
|---|---|---|
| Ingredient | grams | grams |
| Flour | 600 | 600 |
| Water | 378 | 378 |
| Yeast, dry | 10.2 | 10.2 |
| Soybean Oil | 18 | 18 |
| Sugar | 30 | 30 |
| Salt | 12 | 12 |
| Ascorbic 100 | 0.06 | 0.06 |
| Protein Variable | 0 | 18 |

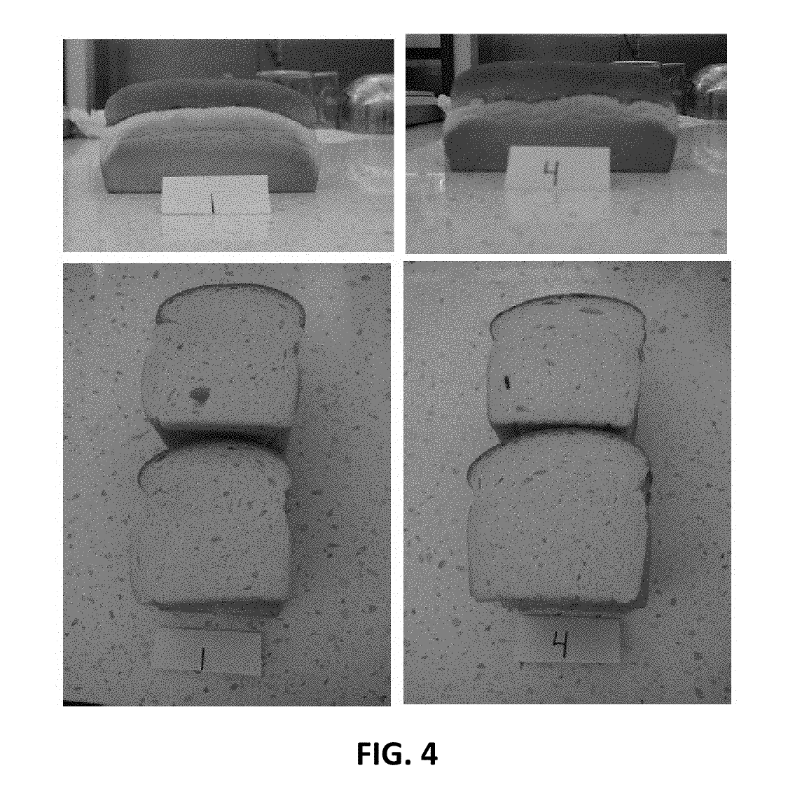

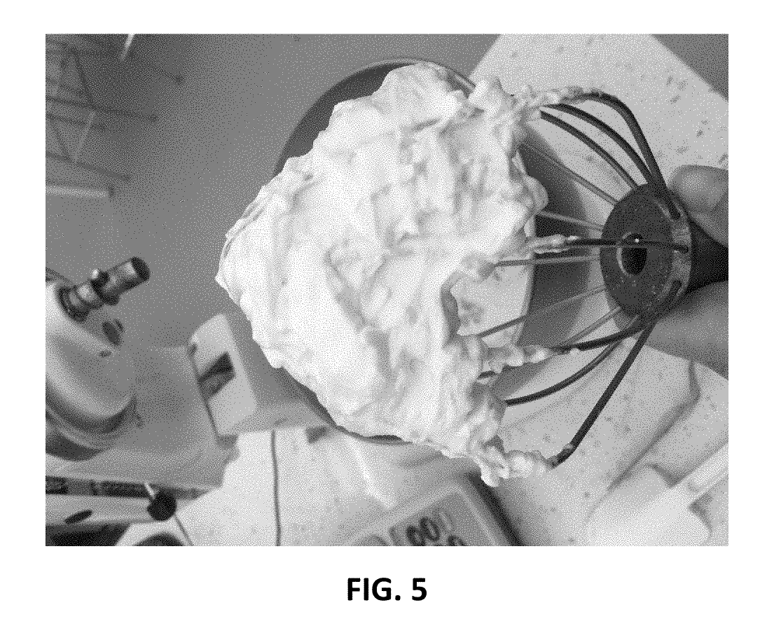

The ingredients were mixed until fully blended, and then the resulting mixture was allowed to rest for 10 minutes. 500 gram samples of each formulation were added to sheets and allowed to rest for an additional 10 minutes. The samples were then added to baking pans, allowed to rest for an additional 50 minutes, and then baked for 19 minutes at 420° F. Photographs of the baked bread products are shown in FIG. 4.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying figures are to be interpreted as illustrative and not limiting.

The constructions described above and illustrated in the figures are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. When introducing elements of the present disclosure or the preferred embodiment(s) thereof, the articles “a”, “an”, “the”, and “said” are intended to mean that there are one or more of the elements. The terms “comprising,” “including,” and “having” are intended to be inclusive, mean that there may be additional elements other than the listed elements, and are used in the sense of “optional” or “may include” and not as “required.” Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A method of preparing a functionalized wheat protein product, the method comprising an enzyme treatment step wherein a wheat protein composition is contacted with a primary enzyme, the primary enzyme comprising a protease that is naturally occurring in a fruit.

2. The method of claim 1, wherein the wheat protein composition has a wheat protein content of at least about 50% by weight.

3. The method of claim 1, wherein the wheat protein composition comprises vital wheat gluten.

4. The method of claim 3, wherein the vital wheat gluten has a wheat protein content of at least about 70% by weight.

5. The method of claim 1, wherein the wheat protein composition comprises a wheat protein isolate.

6. The method of claim 5, wherein the wheat protein isolate has a wheat protein content of at least about 90% by weight.

7. The method of claim 1, wherein the primary enzyme further comprises at least one enzyme selected from the group consisting of bromelain, papain, and actinidin.

8. The method of claim 1, wherein the primary enzyme further comprises bromelain.

9. The method of claim 1, wherein the enzyme treatment step further comprises contacting the wheat protein composition with a secondary enzyme.

10. The method of claim 9, wherein the secondary enzyme comprises a protease.

11. The method of claim 1 further comprising a dilution step, prior to the enzyme treatment step, in which the wheat protein composition is mixed with water.

12. The method of claim 1, wherein the enzyme treatment step is carried out at a temperature of at least about 40° C.

13. The method of claim 1, wherein the enzyme treatment step is carried out for a period of at least about 15 minutes.

14. The method of claim 1, wherein a reaction mixture comprising the primary enzyme and the wheat protein composition is mixed during at least a portion of the enzyme treatment step.

15. The method of claim 14, wherein the enzyme treatment step comprises maintaining the pH of the reaction mixture within a range of from about 3 to about 7.

16. The method of claim 1 further comprising:
a dilution step; and
an enzyme deactivation step, following completion of the enzyme treatment step, in which the primary enzyme and a secondary enzyme are substantially deactivated.

17. The method of claim 16, wherein the deactivation step comprises heating a reaction mixture comprising the primary enzyme and the wheat protein composition to a temperature of at least about 55° C.

18. The method of claim 17, wherein the deactivation step is carried out for a period of at least about 2 minutes.

19. The method of claim 1 further comprise a drying step, following completion of the enzyme treatment step, in which a reaction mixture comprising the primary enzyme and the wheat protein is dried.

20. A functionalized wheat protein product prepared according to the method of claim 1.
